(19) United States
(12) Patent Application Publication
(10) Pub. No.: US 2019/0056438 A1
(43) Pub. Date: Feb. 21, 2019

(54) ADAPTIVE LEARNING BASED ON ELECTROENCEPHALOGRAPHIC DATA

(71) Applicant: Colossio, Inc., Chicago, IL (US)

(72) Inventor: Joseph A. Jaroch, Chicago, IL (US)

(21) Appl. No.: 15/679,764

(22) Filed: Aug. 17, 2017

(51) Int. Cl. G01R 23/00; A61B 5/00; A61B 5/0482; A61B 5/048; A61B 5/0484; G06F 17/30; G09B 19/00

(52) U.S. Cl. CPC G01R 23/005; A61B 5/0006; A61B 5/0482; G09B 19/00; A61B 5/04842; A61B 5/04845; G06F 17/30353; A61B 5/048

(57) ABSTRACT

Systems and a method for adaptive learning style curriculum tailoring through electroencephalography (EEG) are provided. In one or more aspects, a system includes one or more devices to capture raw EEG data associated with one or more first persons attending an information exchange session. A first processor can perform a first processing of the captured raw EEG data to generate EEG frequency data. A communication circuit communicates the EEG frequency data to a central processor. The central processor can process the EEG frequency data to generate a feedback related to attentiveness of the one or more first persons. The feedback can enable a second person to adapt one or more materials presented in the information exchange session based on the feedback for enhanced audience attentiveness.

### TECHNICAL FIELD

The present disclosure generally relates to educational tools and more specifically relates to an adaptive learning through electroencephalography.

### BACKGROUND

Standard classroom education has remained largely unchanged for hundreds of years, focused on the model of a teacher functioning as the orator from whom knowledge is disseminated to listening students. A growing body of research results has shown that this method of teaching is not necessarily best tailored to each individual, and a wide range of preferred learning styles may be available. Therefore, a variety of applications and courses have been developed with the intention to better fit the curriculum to the students by using new approaches including, for example, digital learning, one-on-one tutoring and group studies.

However, it has proven difficult to ascertain the effectiveness of these varied approaches or to compare their effectiveness and efficiency to other methods. In current approaches, test scores are used to indicate the success in learning, which is considered a delayed feedback and can be affected by a variety of different factors, resulting in ignoring the potential of a fundamental mismatch between different teaching styles. Therefore, the ability to provide real-time feedback of the student's level of uptake of information is substantially beneficial. Preferred learning styles may change over time or vary by subject and constant assessment of the effectiveness of education can streamline the teaching process. The streamlining can be achieved by reducing duplicate efforts and identifying instances in which students are failing to learn material at an adequate pace.

### SUMMARY

The disclosed system and methods are provided for correlating the attentiveness of a number of persons learning new material to the material being taught. The disclosed solution can refine and tailor the method of teaching to align with subconscious preferences of the student. The subject technology provides a method of identifying the level of attentiveness of a student through the use of non-invasive means such as electroencephalography (EEG), including EEG implemented through a device (e.g., an EEG headset).

According to certain aspects of the present disclosure, a system for adaptive learning style curriculum tailoring based on EEG data includes one or more devices to capture raw EEG data associated with one or more first persons attending an information exchange session. A first processor can perform a first processing of the captured raw EEG data to generate EEG frequency data. A communication circuit communicates the EEG frequency data to a central processor. The central processor can process the EEG frequency data to generate a feedback related to attentiveness of the one or more first persons. The feedback can enable a second person to adapt one or more materials presented in the information exchange session based on the feedback for enhanced audience attentiveness.

According to certain aspects of the present disclosure, a method of adaptive learning style curriculum tailoring based on EEG data includes providing one or more devices to capture data associated with one or more first persons attending an information exchange session conducted by a second person presenting presentation materials. A first processor can perform a first processing of the captured data to generate a first processed data. The first processed data and the presentation materials are timestamped. The timestamped first processed data and the timestamped presentation materials are communicated to a central processor. The central processor further processes the first processed data to generate a feedback related to attentiveness of the one or more first persons. The captured data includes at least one of raw EEG data, skin conductivity data, or heart rate data. The feedback is a real-time feedback that can enable the second person to adjust conducting of the information exchange session based on the real-time feedback to enhance audience attentiveness.

According to certain aspects of the present disclosure, a system for adaptive learning style curriculum tailoring based on EEG data includes one or more devices configured to capture data associated with one or more first persons attending an information exchange session. A first processor performs a first processing of the captured data to generate a first processed data. A communication device can communicate the first processed data to a central processor. The captured data includes at least one of raw EEG data, skin conductivity data, or heart rate data. The central processor can further process the first processed data to generate a feedback related to attentiveness of the one or more first persons. The feedback may be a real-time feedback that can enable a second person to adjust a presentation of the materials in the information exchange session based on the real-time feedback to enhance audience attentiveness.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 illustrates an example environment in which the subject technology is used.

FIG. 2 is a block diagram illustrating an example architecture of a system for adaptive learning style curriculum tailoring based on electroencephalography (EEG) data, according to certain aspects of the disclosure.

FIG. 3 is a flow diagram illustrating an example process for adaptive learning style curriculum tailoring based on EEG data, according to certain aspects of the disclosure.

FIG. 4 is a diagram illustrating an example information exchange session, in which the subject technology is implemented.

FIG. 5 is a chart illustrating a plot of an example of attentiveness versus time for an attendee of the information exchange session of FIG. 4, according to certain aspects of the disclosure.

FIG. 6 is a chart illustrating a plot of an example of aggregate attentiveness scores versus time for the information exchange session of FIG. 4, according to certain aspects of the disclosure.

FIG. 7 is a chart illustrating plots of an example aggregate attentiveness scores versus time for a department, according to certain aspects of the disclosure.

FIG. 8 is a flow diagram illustrating an example method of providing adaptive learning style curriculum tailoring based on EEG data, according to certain aspects of the disclosure.

FIG. 9 is a block diagram illustrating an example computer system with which certain aspects of the subject technology can be implemented.

In one or more implementations, not all of the depicted components in each figure may be required, and one or more implementations may include additional components not shown in a figure. Variations in the arrangement and type of the components may be made without departing from the scope of the subject disclosure. Additional components, different components, or fewer components may be utilized within the scope of the subject disclosure.

### DETAILED DESCRIPTION

The detailed description set forth below is intended as a description of various implementations and is not intended to represent the only implementations in which the subject technology may be practiced. As those skilled in the art would realize, the described implementations may be modified in various different ways, all without departing from the scope of the present disclosure. Accordingly, the drawings and description are to be regarded as illustrative in nature and not restrictive.

### General Overview

This subject technology provides a method and a system for identifying the level of attentiveness of a student through the use of a non-invasive electroencephalography (EEG) device. The EEG device can be any device (e.g., an EEG headset) capable of capturing EEG data and providing raw EEG data. In some implementations, the EEG device can perform a discrete Fourier transformation analysis to identify the frequency ranges of captured raw EEG data including brainwaves. The identified frequency ranges can be used to establish and monitor the types of waves produced by the brain. The identified brain waves, for example, may indicate concentration (e.g., beta and/or gamma waves) or relaxation or drifting focus (e.g., alpha waves). In some implementations, the captured brain waves can be recorded, buffered and transmitted to a central aggregator.

The central aggregator can reside within the cloud, in one or more implementations, to allow for correlation across groups of students or attendees. The cloud can provide an easy access from a variety of devices, as well as having simpler setup and initialization steps. Cloud access obviates the need for configuring local policies within a local network (e.g., a local area network (LAN), a personal area network (PAN)), a campus area network (CAN), a metropolitan area network (MAN), a wide area network (WAN), a virtual private network (VPN) or other networks).

When there are concerns or issues with collecting brainwave frequency ranges, skin conductivity and heart rate can be used as a rough means for identifying attentiveness. This allows for coverage in circumstances where EEGs are deemed too invasive. Additionally, the subject disclosure provides a method to correlate and collect the overall brainwave frequency ranges and to associate them with the material (e.g., audio, text or video material) being spoken, displayed, presented or conveyed using any other means from a teacher, an education platform or a software application.

In some implementations, feedback is provided to the teacher, the education platform or the software application of an aggregate and personal mental state of the students or users. The feedback can be used to focus and facilitate the adjustment of the approach being used to convey the information (e.g., of the material). A primary use-case for the technology is to aid teachers in ensuring their lessons are resonating properly with students. This can be especially beneficial in cases with increased class sizes where individual attention of the students is harder to manage.

Another use-case is in focus groups to measure the effectiveness of content, especially new product releases and other demos. Real-time feedback can be correlated while instruction and information dissemination is in-process, allowing the presenter to change their tactics or to review the results after the session has completed.

### Example System Architecture

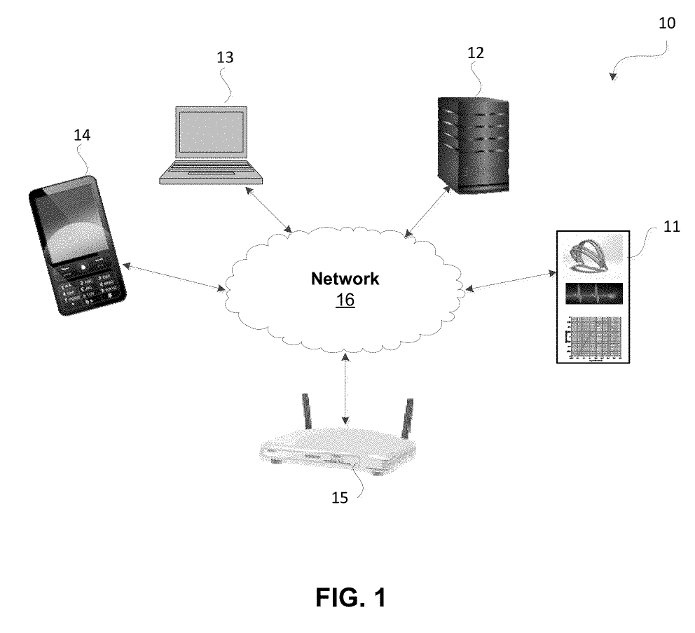

FIG. 1 illustrates an example environment in which the subject technology is used. The architecture 10 includes one or more devices 11, a central processor (e.g. a server) 12, a computing device 13, a portable communication device 14 and an access point 15 communicating (e.g., wirelessly) over a network 16. Examples of the one or more devices 11 may include, but are not limited to, an electroencephalography (EEG) headset, a skin conductivity measurement device and a heart rate measurement device. In one or more aspects, the EEG headset can capture raw EEG data associated with one or more first persons (e.g., as one or more students, trainees, application users or online users) of an information exchange session (e.g., a class room, a seminar, a conference, a training session, an application or an on-line session). In some implementations, the EEG headset may include a module that can perform first processing of the raw EEG data, for example, by performing Fourier transform analysis. In some implementations, the skin conductivity measurement device and a heart rate measurement device are integrated with a smart device such as a smart phone or a smart watch of the attendees of the information exchange session (hereinafter “the session”).

In some implementations, the central processor 12 is a local server or a cloud server capable of cloud computing. The computing device 13 may be a personal computer such as a laptop computer and the portable communication device 14 may be a smart phone or a personal digital assistant (PDA) of the first person (hereinafter “the attendee”) or the second person (e.g., a teacher, a trainer, a lecturer, a speaker or a presenter; hereinafter “presenter”). The computing device 13 and/or the portable communication device 14 may have the presentation material such as Power Point presentation (PPP) material or other text or media files (e.g., audio or video files) stored on them or be able to download the presentation material. The computing device 13 and/or the portable communication device 14 may be used to present (e.g., display) the presentation material to the attendees, for example, via a suitable screen. The access point 15 may be a wireless access point that facilitates communication, via the network 16, of the devices 11, central processor 12, computing device 13 and the portable communication device 14.

Examples of the network 16 include any one or more of a personal area network (PAN), a local area network (LAN), a campus area network (CAN), a metropolitan area network (MAN), a wide area network (WAN), a virtual private network (VPN), a broadband network (BBN), the Internet and the like. Further, the network 16 can include, but is not limited to, any one or more of the following network topologies, including a bus network, a star network, a ring network, a mesh network, a star-bus network, a tree or hierarchical network and the like.

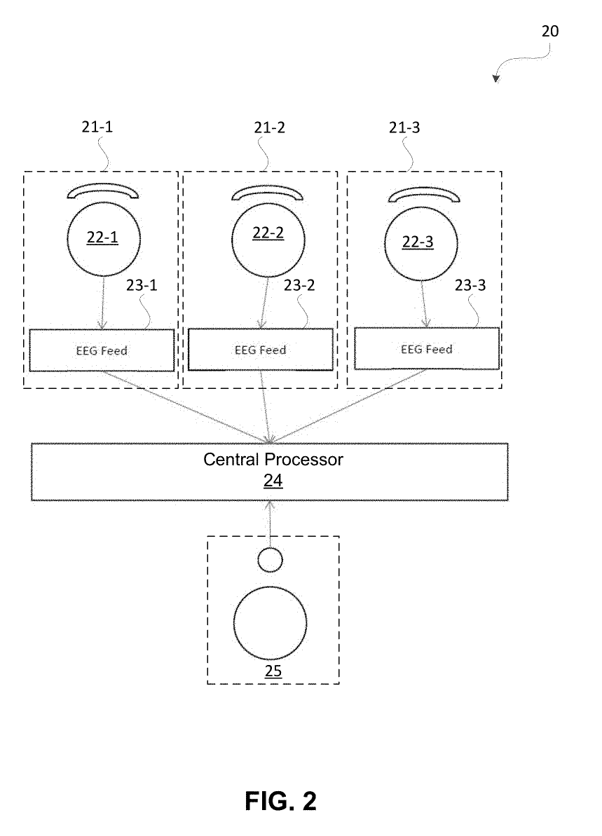

FIG. 2 is a block diagram illustrating an example architecture of a system 200 for adaptive learning style curriculum tailoring based on EEG data, according to certain aspects of the disclosure. The system 200 includes a number of data collection devices 21 (e.g., 21-1, 21-2 and 21-3). The number of data collection devices 21 may be equal to the number of attendees of the session such that each attendee can use at least one of the data collection devices 21. In some implementations, the data collection devices 21 are EEG headsets 22 (e.g., 22-1, 22-2 and 22-3). The EEG headsets 22 may include EEG feeds 23 (e.g., 23-1, 23-2 and 23-3). In some aspects, the EEG feeds 23 may be or may include communication interfaces such as wireless communication interfaces that can communicate with the network 16 of FIG. 1. In some aspects, the raw EEG data includes brain waves that can be represented by different frequency ranges including categories such as Delta waves (within a frequency range of about 0.2-3 Hz), Theta waves (within a frequency range of about 4-7 Hz), Alpha waves (within a frequency range of about 8-13 Hz), Mu waves (within a frequency range of about 7.5-12.5 Hz), Sensorimotor Rhythm (SMR) waves (within a frequency range of about 12.5-15.5 Hz), Beta waves (within a frequency range of about 16-31 Hz) and Gamma waves (within a frequency range of about 32-100 Hz).

In some implementations, the EEG feeds 23 may include one or more local processors capable of performing a first processing on the collected EEG raw data. The first processing may include performing a discrete Fourier transformation analysis such as a fast Fourier transform (FFT) on the collected EEG raw data to generate EEG frequency data. In some aspects, the first processing may include filtering (e.g., using digital filter) of EEG frequency data or the raw data to remove noise. In one or more implementations, rather than forcing collected brainwaves into the above mentioned categories, a variation (delta) over time is determined by looking at all possible ranges of EEG frequency data and studying variation with time of each band range. Because the analysis is applied on a per-attendee basis, specific averages over time and trends can be spotted, which could be missed with generalizations. For example, if one attendee's baseline Alpha brainwaves tend to be at 12 Hz, and another attendee's Alpha brainwaves tend to be at 8 Hz, a simple aggregated view may lose the individualized variances. However, if each attendee's Alpha brainwave increases by 10% (e.g., to 13.2 Hz and 8.8 Hz, respectively), the delta may become meaningful. Additionally, for attendees whose brainwaves do not precisely match conventional ranges, by using the delta in readings rather than the absolute readings, variability from the normal condition can be eliminated.

The collected raw data or processed data after FFT and/or filtering may be time stamped and communicated to the central processor 12 (e.g., a local server or a cloud server) for further processing. In some implementations, the central processor 12 may time stamp, upon receiving, the collected raw data or the processed data (e.g., after FFT and/or filtering).

In some aspects, the presenter 25 may upload (e.g., in real-time) the presentation material from the computing device 13 or the portable communication device 14 to the central processor 12. The presentation material may be time stamped by the computing device 13 or the portable communication device 14 upon transmission to the central processor 12. In some aspects, the central processor 12 may time stamp, upon receiving, the presentation material. The central processor 12 can process the EEG frequency data to generate a feedback related to attentiveness of the attendees. The feedback can enable the presenter to adapt one or more materials presented in the session based on the feedback to achieve enhanced audience attentiveness and to ensure the lessons (e.g., presentations) are resonating properly with attendees, especially in cases with increased class sizes where individual attention is harder to manage. Another use-case of the subject technology is in focus groups for measuring the effectiveness of content, especially new product releases and other demos. Real-time feedback can be correlated while instruction and information dissemination is in-process, allowing the presenter to change their tactics and/or to review the results after the session has completed.

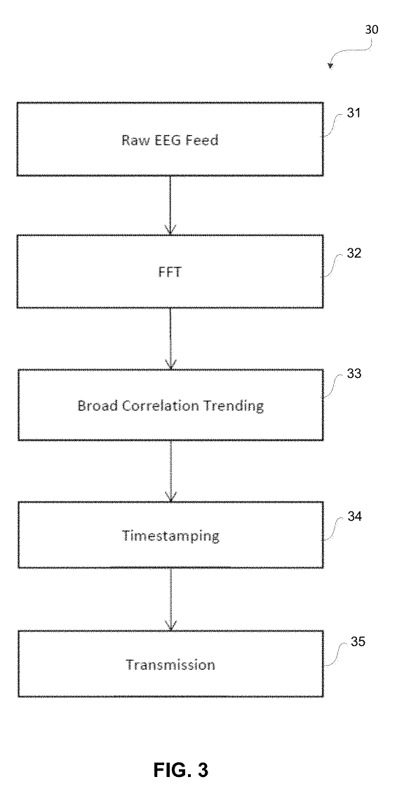

FIG. 3 is a flow diagram illustrating an example process 30 for adaptive learning style curriculum tailoring based on EEG data, according to certain aspects of the disclosure. The process 30 begins with operation block 31, where EEG raw data is collected, for example, by EEG headsets 22 of FIG. 2. The EEG raw data is converted to EEG frequency data, at operation block 32, by a local processor, for example, of the EEG Feed 23. At operation block 33, a broad correlation trending may be performed, for example, to correlate each attendee's EEG frequency data to one of the brainwave categories. At operation block 34, the correlated data is time stamped by the local processor of the EEG Feed 23. The time stamped data is then transmitted, at operation block 35, to the central processor (e.g., processor 12 of FIG. 1) for further processing and generating feedback that is an indication of each attendee's attentiveness.

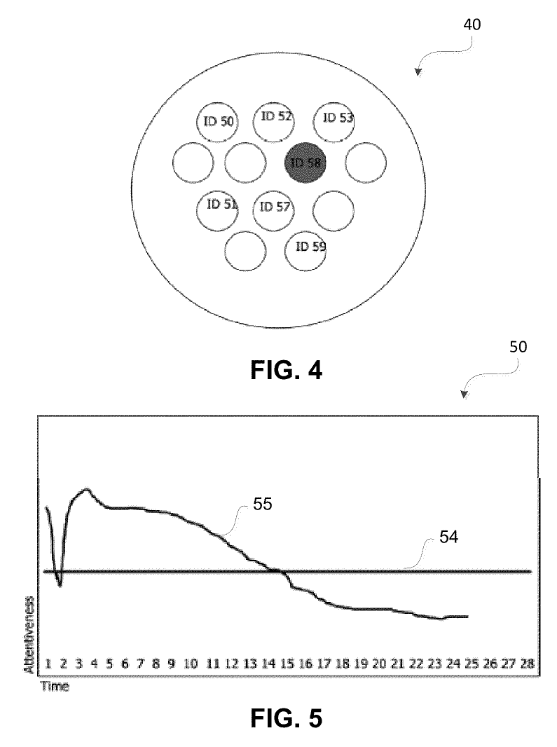

FIG. 4 is a diagram illustrating an example information exchange session 40, in which the subject technology is implemented. The information exchange session 40 can be a class room, a seminar, a conference, a training session or an on-line session with a number of attendees. Each attendee may be assigned an identification number (ID) (e.g., ID 50, ID 51 . . . ID 59) that can be used in communication and storage of data associated with each attendee. For example, for each attendee, the stored brainwaves, the prepared feedback including the attentiveness of the attendee can be labeled with the corresponding ID of the attendee.

FIG. 5 is a chart 50 illustrating a plot of an example of attentiveness 55 versus time for an attendee of the information exchange session of FIG. 4, according to certain aspects of the disclosure. In some aspects, the central processor (e.g., processor 12 of FIG. 1) can correlate time stamped attendee's processed brain wave data, skin conductivity data and/or heart rate data with an attentiveness score that can vary with time, as shown in the plot of the attentiveness 55 of the chart 50 versus time (in minutes). The plot of the attentiveness 55 shown in the chart 50 may correspond to an attendee with an ID, for example, ID 58. The attentiveness score is seen to change around an expected score designated by the line 54. The attendee's attentiveness score initially rises within a time window of about 2-4 minutes and decreases afterwards. The attentiveness score is falling below the expected score after about 14 minutes. The attentiveness score along with the attendee's ID number (e.g., ID 58) can be feedback to the presenter to watch the attendee 58 (e.g., with ID 58) and try to attract attention of that attendee to the presentation material, for example by posing a question or other ways. In some aspects, the expected score designated by the line 54 can be determined based on historic data collected from the attendee 58 or from an entire class of attendees. The attentiveness scores of individual attendees can be buffered and fed back to the presenter, in real time and/or after the end of the session, along with other session data such as time stamped presentation material. The data associated with the attentiveness scores of individual attendees may be converted to respective plots for display on a display device, for example, of a computing device (e.g., 13 of FIG. 1) and/or a potable communication device (e.g., 14 of FIG. 1). The data associated with the attentiveness scores of individual attendees can be stored in memory (e.g., non-volatile memory, such as a cloud storage) coupled to the central processor (e.g., 12 of FIG. 1) for post-lesson analysis to allow the presenter to continue without distractions and retroactively adjust presentation material and presenting approaches for subsequent sessions.

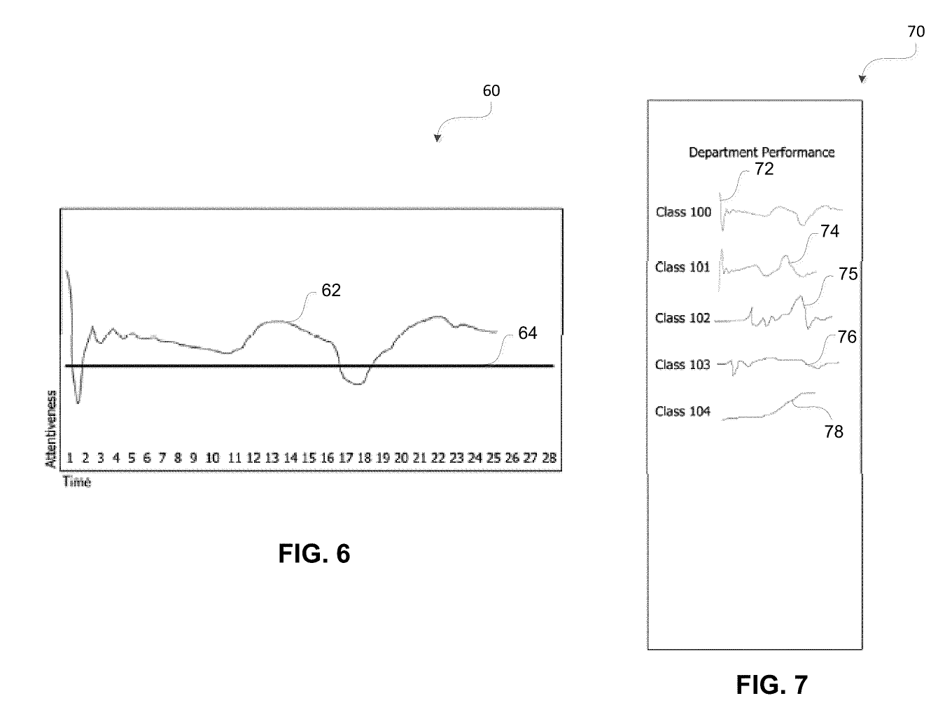

FIG. 6 is a chart 60 illustrating a plot of an example of aggregate attentiveness scores 62 versus time for the information exchange session of FIG. 4, according to certain aspects of the disclosure. The aggregate attentiveness scores 62 versus time (e.g., minutes) can be determined based on aggregation of attentiveness scores (e.g., ID 55 of FIG. 5) of the entire attendees of the information exchange session (e.g., a classroom) of FIG. 4. (e.g., attendees with ID 51, ID 52 . . . ID 59). The aggregate attentiveness scores 62 is seen to fluctuate, but for most of the presentation time is above an expected session attentiveness score 64, and goes below the expected session attentiveness score 64 for only a short time window of about 2 minutes (e.g., between about 16.5 to 18.5 minutes). The aggregate attentiveness scores 62 can be buffered and fed back to the presenter, in real time and/or after the end of the session, along with other session data such as time stamped presentation material. The data associated with aggregate attentiveness scores 62 may be converted to a respective plot for display on a display device, for example, of a computing device (e.g., device 13 of FIG. 1) and/or a portable communication device (e.g., device 14 of FIG. 1). The data associated with the aggregate attentiveness scores 62 can be stored in memory (e.g., non-volatile memory, such as a cloud storage) coupled to the central processor (e.g., processor 12 of FIG. 1) for post-lesson analysis to allow the presenter to continue without distractions and retroactively adjust presentation material and presenting approaches for subsequent sessions.

FIG. 7 is a chart 70 illustrating plots of example aggregate attentiveness scores versus time for a department, according to certain aspects of the disclosure. The aggregation of attentiveness data may be performed for an entire department, for example, including a number of class rooms (e.g., class 100, class 101 . . . class 104). The attentiveness plots 72, 74, 75, 76 and 78 show attentiveness scores for classes 100, 101, 102, 103 and 104, for example, correlated across different instructors and in a setting-agnostic manner. The data associated with attentiveness plots 72, 74, 75, 76 and 78 can be buffered and fed back to the presenter, in real time and/or after the end of the session, along with other session data such as time stamped presentation material. The data associated with attentiveness plots 72, 74, 75, 76 and 78 may be converted to respective plots for display on a display device, for example, of a computing device (e.g., device 13 of FIG. 1) and/or a portable communication device (e.g., device 14 of FIG. 1). The data associated with the attentiveness plots 72, 74, 75, 76 and 78 can be stored in memory (e.g., non-volatile memory, such as a cloud storage) coupled to the central processor (e.g., processor 12 of FIG. 1) for post-lesson analysis to allow the presenters to continue without distractions and retroactively adjust presentation materials and presenting approaches for subsequent sessions.

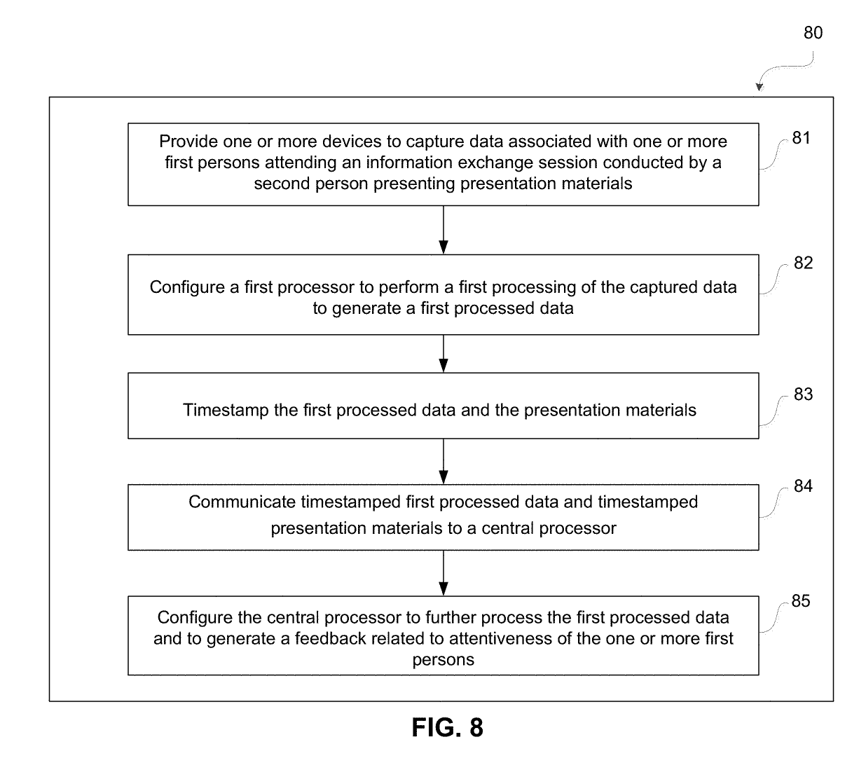

FIG. 8 is a flow diagram illustrating an example method 80 of providing adaptive learning style curriculum tailoring based on EEG data (e.g., provided by device 11 of FIG. 1), according to certain aspects of the disclosure. The method 80 includes providing one or more devices (e.g., device 11 of FIG. 1) to capture data associated with one or more first persons attending an information exchange session conducted by a second person (e.g., presenter 25 of FIG. 2) presenting presentation materials (81). A first processor (e.g., included in device 11 of FIG. 1 or EEG feed 23 of FIG. 2) performs a first processing of the captured data to generate a first processed data (82). The first processed data and the presentation materials are timestamped (e.g., by device 11 or processor 12 of FIG. 1) (83). The timestamped first processed data and the timestamped presentation materials are communicated (e.g., via the network 16 of FIG. 4) to a central processor (e.g., processor 12 of FIG. 1 or processor 24 of FIG. 2) (84). The central processor further processes the first processed data to generate a feedback (e.g., ID 55 of FIG. 5 or ID 62 of FIG. 6) related to attentiveness of the one or more first persons (85). The captured data includes at least one of raw EEG data, skin conductivity data or heart rate data. The feedback may be a real-time feedback that can enable the second person to adjust conducting of the information exchange session based on the real-time feedback to enhance audience attentiveness.

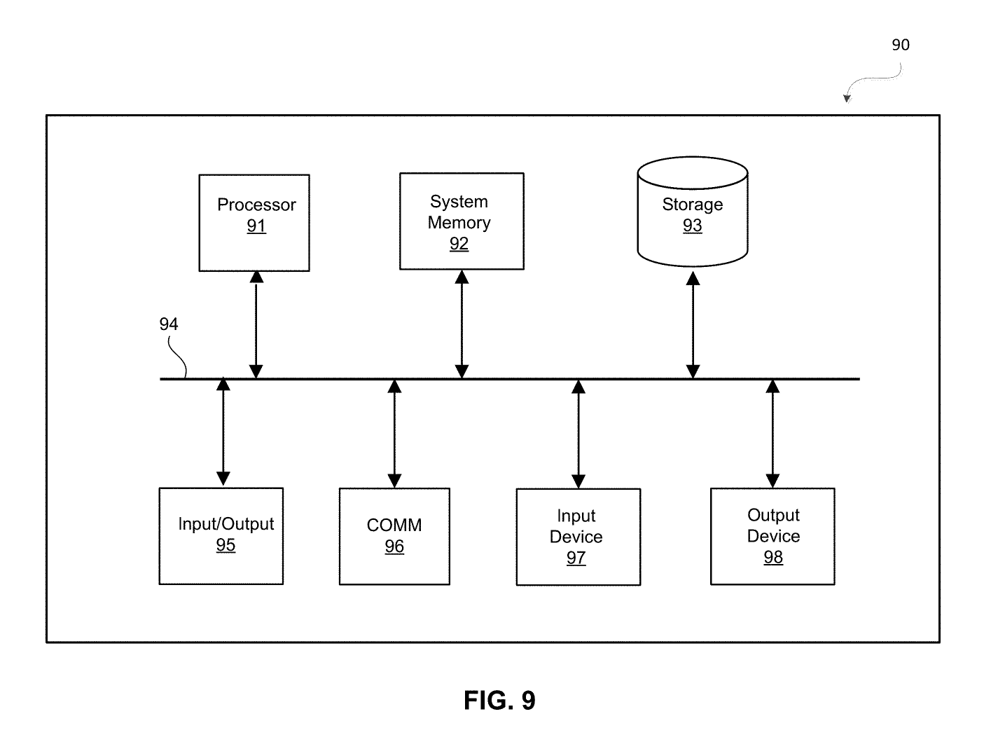

FIG. 9 is a block diagram illustrating an example computer system with which certain aspects of the subject technology can be implemented. In some aspects, the computer system 90 may represent the server 12, the computing device 13 and/or the mobile device 14 of FIG. 1. In certain aspects, the computer system 90 may be implemented using hardware or a combination of software and hardware, either in a dedicated server or integrated into another entity or distributed across multiple entities.

Computer system 90 (e.g., processor 12, the computing device 13 or the portable communication device 14) includes a bus 94 or other communication mechanism for communicating information and a processor 91 coupled with bus 94 for processing information. According to one aspect, the computer system 90 can be a cloud computing server of an infra-structure-as-a-service (IaaS) and can be able to support platform-as-a-service (PaaS) and software-as-a-service (SaaS).

Computer system 90 can include, in addition to hardware, code that creates an execution environment for the computer program in question, e.g., code that constitutes processor firmware, a protocol stack, a database management system, an operating system, or a combination of one or more of them stored in an included memory 92, such as a Random Access Memory (RAM), a flash memory, a Read Only Memory (ROM), a Programmable Read-Only Memory (PROM), an Erasable PROM (EPROM), registers, a hard disk, a removable disk, a CD-ROM, a DVD, or any other suitable storage device, coupled to bus 94 for storing information and instructions to be executed by processor 91. The processor 91 and the memory 92 can be supplemented by, or incorporated in, special purpose logic circuitry.

The instructions may be stored in the memory 92 and implemented in one or more computer program products, i.e., one or more modules of computer program instructions encoded on a computer readable medium for execution by, or to control the operation of, the computer system 90, and according to any method well known to those of skill in the art

A computer program as discussed herein does not necessarily correspond to a file in a file system. A program can be stored in a portion of a file that holds other programs or data (e.g., one or more scripts stored in a markup language document), in a single file dedicated to the program in question or in multiple coordinated files (e.g., files that store one or more modules, subprograms, or portions of code). A computer program can be deployed to be executed on one computer or on multiple computers that are located at one site or distributed across multiple sites and interconnected by a communication network. The processes and logic flows described in this specification can be performed by one or more programmable processors executing one or more computer programs to perform functions by operating on input data and generating output.

Computer system 90 further includes a data storage device 93 such as a magnetic disk or optical disk, coupled to bus 94 for storing information and instructions. Computer system 90 may be coupled via input/output module 95 to various devices. The input/output module 95 can be any input/output module. Example input/output modules 95 include data ports such as USB ports. In addition, input/output module 95 may be provided in communication with processor 91, so as to enable near area communication of computer system 90 with other devices. The input/output module 95 may provide, for example, for wired communication in some implementations or for wireless communication in other implementations, and multiple interfaces may also be used. The input/output module 95 is configured to connect to a communications module 96. Example communications modules 96 may include networking interface cards, such as Ethernet cards and modems.

In certain aspects, the input/output module 95 is configured to connect to a plurality of devices, such as an input device 97 and/or an output device 98. Example input devices 97 include a keyboard and a pointing device, e.g., a mouse or a trackball, by which a user can provide input to the computer system 90. Other kinds of input devices 97 can be used to provide for interaction with a user as well, such as a tactile input device, visual input device, audio input device or brain-computer interface device.

According to one aspect of the present disclosure, at least portions of the process 30 and the method 80 can be implemented using a computer system 90 in response to processor 91 executing one or more sequences of one or more instructions contained in memory 92. Such instructions may be read into memory 92 from another machine-readable medium, such as data storage device 93. Execution of the sequences of instructions contained in main memory 92 causes processor 91 to perform the process steps described herein. One or more processors in a multi-processing arrangement may also be employed to execute the sequences of instructions contained in memory 92. In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back end component, e.g., such as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware or front end components.

In one aspect, a method may be an operation, an instruction or a function and vice versa. In one aspect, a clause or a claim may be amended to include some or all of the words (e.g., instructions, operations, functions or components) recited in other one or more clauses, one or more words, one or more sentences, one or more phrases, one or more paragraphs and/or one or more claims.

As used herein, the phrase “at least one of” preceding a series of items, with the terms “and” or “or” to separate any of the items, modifies the list as a whole, rather than each member of the list (i.e., each item). The phrase “at least one of” does not require selection of at least one item; rather, the phrase allows a meaning that includes at least one of any one of the items, and/or at least one of any combination of the items and/or at least one of each of the items. By way of example, the phrases “at least one of A, B, and C” or “at least one of A, B, or C” each refer to only A, only B, or only C; any combination of A, B, and C; and/or at least one of each of A, B, and C.

Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

A reference to an element in the singular is not intended to mean “one and only one” unless specifically stated, but rather “one or more.” Underlined and/or italicized headings and subheadings are used for convenience only, do not limit the subject technology, and are not referred to in connection with the interpretation of the description of the subject technology. Relational terms such as first and second and the like may be used to distinguish one entity or action from another without necessarily requiring or implying any actual such relationship or order between such entities or actions. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description. No claim element is to be construed under the provisions of 35 U.S.C. § 112, sixth paragraph, unless the element is expressly recited using the phrase “means for” or, in the case of a method claim, the element is recited using the phrase “step for”.

While this specification contains many specifics, these should not be construed as limitations on the scope of what may be claimed, but rather as descriptions of particular implementations of the subject matter. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

The subject matter of this specification has been described in terms of particular aspects, but other aspects can be implemented and are within the scope of the following claims. For example, while operations are depicted in the drawings in a particular order, this should not be understood as requiring that such operations be performed in the particular order shown or in sequential order, or that all illustrated operations be performed, to achieve desirable results. The actions recited in the claims can be performed in a different order and still achieve desirable results. As one example, the processes depicted in the accompanying figures do not necessarily require the particular order shown, or sequential order, to achieve desirable results. In certain circumstances, multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products.

The title, background, brief description of the drawings, abstract and drawings are hereby incorporated into the disclosure and are provided as illustrative examples of the disclosure, not as restrictive descriptions. It is submitted with the understanding that they will not be used to limit the scope or meaning of the claims. In addition, in the detailed description, it can be seen that the description provides illustrative examples and the various features are grouped together in various implementations for the purpose of streamlining the disclosure. The method of disclosure is not to be interpreted as reflecting an intention that the claimed subject matter requires more features than are expressly recited in each claim. Rather, as the claims reflect, inventive subject matter lies in less than all features of a single disclosed configuration or operation. The claims are hereby incorporated into the detailed description, with each claim standing on its own as a separately claimed subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirements of the applicable patent law, nor should they be interpreted in such a way.

What is claimed is:

1. A system for adaptive learning style curriculum tailoring based on electroencephalography (EEG) data, the system comprising:
one or more devices configured to capture raw EEG data associated with one or more first persons attending an information exchange session;
a first processor configured to perform a first processing of the captured raw EEG data to generate EEG frequency data; and
a communication circuit configured to communicate the EEG frequency data to a central processor,
wherein:
the central processor is configured to process the EEG frequency data and to generate a feedback related to attentiveness of the one or more first persons, and
the feedback is configured to enable a second person to adapt one or more materials presented in the information exchange session based on the feedback for an enhanced audience attentiveness.

2. The system of claim 1, wherein the one or more devices comprise one or more EEG headset.

3. The system of claim 1, wherein the information exchange session comprises at least one of a teaching session, a training session, a seminar, a conference, an application or an online course, and wherein the one or more first persons comprise one or more students, trainees, attendees, application users or online users.

4. The system of claim 1, wherein the second person comprises one of a teacher, a trainer, a lecturer, a speaker, or a presenter.

5. The system of claim 1, wherein the first processing comprises a discrete Fourier transformation analysis, and the raw EEG data comprises brain waves including, Delta, Theta, Alpha, Mu, sensorimotor rhythm (SMR), Beta and Gamma waves, which are represented by different frequency ranges in the EEG frequency data.

6. The system of claim 1, wherein the first processor is integrated with the one or more devices.

7. The system of claim 1, wherein the central processor comprises a cloud processor configured to receive the EEG frequency data and to send the feedback over the Internet.

8. The system of claim 1, wherein the one or more devices are configured to communicate over the Internet.

9. The system of claim 1, wherein the communication circuit is configured to transmit, in real time, the one or more materials presented in the information exchange session including text, audio and video data to the central processor.

10. The system of claim 9, wherein the first processor is configured to timestamp the one or more materials presented in the information exchange session and the generated EEG frequency data before transmission to the central processor.

11. The system of claim 1, wherein the central processor is configured to timestamp upon receipt the one or more materials presented in the information exchange session and the generated EEG frequency data.

12. The system of claim 11, wherein the central processor is configured to correlate at least portions of the generated EEG frequency data to the one or more materials presented in the information exchange session based on timestamp information, wherein the portions of the generated EEG frequency data correspond to various states of attentiveness of the first person.

13. The system of claim 12, wherein the feedback comprises a real-time feedback, and wherein the feedback is based on correlation of the various states of attentiveness of the first person and the one or more materials presented in the information exchange session.

14. The system of claim 1, further comprising a skin conductivity and heart rate measurement device configured to perform real-time skin conductivity and heart rate measurement of the first person, to generate skin conductivity and heart rate data, wherein the generated skin conductivity and heart rate data are timestamped.

15. The system of claim 14, wherein the central processor is configured to analyze the timestamped skin conductivity and heart rate data and identify portions of timestamped skin conductivity and heart rate data that correspond to various states of attentiveness of the first person.

16. The system of claim 15, wherein the central processor is configured to correlate the portions of timestamped skin conductivity and heart rate data that correspond to various states of attentiveness of the first person with the one or more materials presented in the information exchange session using timestamp information to generate the feedback.

17. A system comprising:
one or more devices configured to capture data associated with one or more first persons attending an information exchange session;
a first processor configured to perform a first processing of the captured data to generate a first processed data; and
a communication device configured to communicate the first processed data to a central processor,
wherein:
the captured data includes at least one of raw electroencephalography (EEG) data, skin conductivity data or heart rate data,
the central processor is configured to further process the first processed data and to generate a feedback related to attentiveness of the one or more first persons, and
the feedback is a real-time feedback configured to enable a second person to adjust a presentation of materials in the information exchange session based on the real-time feedback to enhance audience attentiveness.

18. The system of claim 17, wherein the communication device is further configured to communicate, in real-time, presentation materials to the central processor, wherein the central processor is configured to timestamp the first processed data and presentation materials, and wherein the central processor is further configured to generate the feedback based on analysis of timestamped first processed data and timestamped presentation materials.

19. A method comprising:
providing one or more devices to capture data associated with one or more first persons attending an information exchange session conducted by a second person presenting presentation materials;
configuring a first processor to perform a first processing of the captured data to generate a first processed data;
timestamping the first processed data and the presentation materials;
communicating timestamped first processed data and timestamped presentation materials to a central processor; and
configuring the central processor to further process the first processed data and to generate a feedback related to attentiveness of the one or more first persons,
wherein:
the captured data includes at least one of raw electroencephalography (EEG) data, skin conductivity data, or heart rate data, and
the feedback comprises a real-time feedback that is configured to enable the second person to adjust conducting of the information exchange session based on the real-time feedback to enhance audience attentiveness.

20. The method of claim 19, further comprising configuring the central processor to generate the feedback based on analysis of the timestamped first processed data and the timestamped presentation materials and to correlate portions of the timestamped first processed data indicating various states of attentiveness of the one or more first persons with corresponding portion of the timestamped presentation materials.
